(19) United States
(12) Patent Application Publication
(10) Pub. No.: US 2014/0200270 A1
(43) Pub. Date: Jul. 17, 2014

(54) VITAMINS C AND K FOR TREATING POLYCYSTIC DISEASES

(71) Applicants: SUMMA HEALTH SYSTEM, Akron, OH (US); MAYO FOUNDATION FOR MEDICAL EDUCATION AND RESEARCH, Rochester, MN (US)

(72) Inventors: Tetyana V. Masyuk, Rochester, MN (US); Nicholas F. Larusso, Rochester, MN (US); James M. Jamison, Stow, OH (US); Thomas M. Miller, El Cajon, CA (US)

(73) Assignees: SUMMA HEALTH SYSTEM, Akron, OH (US); MAYO FOUNDATION FOR MEDICAL EDUCATION AND RESEARCH, Rochester, MN (US)

(21) Appl. No.: 14/152,710

(22) Filed: Jan. 10, 2014

Related U.S. Application Data
(60) Provisional application No. 61/751,702, filed on Jan. 11, 2013.

(51) Int. Cl. A61K 31/375; A61K 31/122

(52) U.S. Cl. CPC A61K 31/375; A61K 31/122
USPC 514/474

(57) ABSTRACT

Provided herein are methods for treating, preventing, or ameliorating one or more symptoms of a polycystic disease in a subject, comprising administering to the subject a therapeutically effective amount of vitamins C and K.

### CROSS REFERENCE TO RELATED APPLICATION

This application claims the benefit of the priority of U.S. Provisional Application No. 61/751,702, filed Jan. 11, 2013; the disclosure of which is incorporated herein by reference in its entirety.

### FIELD

Provided herein are methods for treating, preventing, or ameliorating one or more symptoms of a polycystic disease in a subject, comprising administering to the subject a therapeutically effective amount of vitamins C and K.

### BACKGROUND

Polycystic kidney disease (PKD) is the most common life-threatening genetic disease, affecting more than 600,000 Americans and an estimated 12.5 million people worldwide. PKD is characterized by the presence of fluid-filled cysts in the kidneys, often resulting in renal failure. About 50 percent of patients with PKD will have kidney failure by age 60 and about 60 percent will have kidney failure by age 70. Although kidneys usually are the most severely affected organs, PKD can cause cysts to develop in the liver and elsewhere in the body. Liver cystogenesis occurs in more that 95% of PKD patients.

Autosomal dominant PKD (ADPKD) and autosomal recessive PKD (ARPKD) are the most common forms of PKD. Harris et al., Annu. Rev. Med. 2009, 60, 321-337. ADPKD is passed from a parent to a child by an autosomal dominant type of inheritance. Thus, only one copy of the abnormal gene is needed to cause the disease. ADPKD is caused by mutations in PKD1 (encoding polycystin-1), PKD2 (encoding polycystin-2), and/or PKD3 (unmapped). Harris et al., Annu. Rev. Med. 2009, 60, 321-337; Hughes et al., Nature Genetics 1995, 10, 151-160; Mochizuki et al., Science 1996, 272, 1339-1342; Koptides et al., Hum. Genet. 2000, 107, 115-126. Polycystin-1 is a membrane receptor capable of binding and interacting with many proteins, carbohydrates, and lipids, and eliciting intracellular responses through phosphorylation pathways, whereas polycystin-2 is thought to act as a calcium-permeable channel. Polycystins regulate tubular and vascular development in the kidneys and in other organs, including the liver, brain, arterial blood vessels, and pancreas, causing extra-renal manifestations of the disease.

ARPKD is the most common genetic cystic renal disease occurring in infancy and childhood. ARPKD is passed by an autosomal recessive pattern of inheritance. Thus, both parents must carry the abnormal gene, and both must pass the gene to the child in order for the child to develop the disease. ARPKD is caused by mutations in PKHD1 (encoding fibrocystin). Harris et al., Annu. Rev. Med. 2009, 60, 321-337; Ward et al., Nature Genetics. 2002, 30, 259-269. Fibrocystin has been found in the same complex as polycystin-2. However, the precise function of fibrocystin is at present unknown, but it may mediate its activity through polycystin-2. Harris et al., Annu. Rev. Med. 2009, 60, 321-337.

Other polycystic diseases include polycystic liver disease (PLD), polycystic pancreas disease (PPD), and polycystic ovarian syndrome (PCOS). Abdul-Majeed et al., Obstet. Gynecol. Int. 2011, Epub 2011. PLD is characterized by the presence of multiple bile duct-derived epithelial cysts scattered in the liver parenchyma. PLD occurs isolated in the liver or in combination with PKD. Though cystic liver is one of the most common extrarenal manifestations observed in PKD, it also exists as an isolated inherited cystic disease without any kidney cysts. Qian, Adv. Chronic Kidney Dis. 2010, 17, 181-189. Isolated autosomal dominant polycystic liver disease (ADPLD) is genetically distinct from PLD associated with ADPKD, although it may have similar pathogenesis and clinical manifestations. Qian et al., Hepatology 2003, 37, 164-171; Reces et al., World J. Gastroenterol. 2005, 11, 7690-7693. PLD is genetically heterogeneous, all being transmitted autosomally in a dominant or recessive fashion. PLD is caused by mutations in PPRKCSH or SEC63. Davila et al., Nature Genetics 2004, 36, 575-577; Waanders et al., Hum. Mutat. 2006, 27, 830. PPRKCSH encodes the noncatalytic β-subunit of glucosidase II (GIIβ) involved in the folding of glycoproteins, whereas SEC63 encodes a protein involved in protein trafficking in the ER. Fedeles et al., Nature Genetics 2011, 43, 639-647; Muller et al., FEBS Lett. 2011, 585, 596-600; Qian, Adv. Chronic Kidney Dis. 2010, 17, 181-189.

Currently, there is no approved treatment for polycystic diseases to halt cyst growth. For example, the management for PKD and PLD is centered on palliating symptoms and treating complications. Qian, Adv. Chronic Kidney Dis. 2010, 17, 181-189. Therefore, there is a need for an effective method for the treatment of a polycystic disease.

### SUMMARY OF THE DISCLOSURE

Provided herein is a method for treating, preventing, or ameliorating a polycystic disease in a subject, comprising administering to the subject a therapeutically effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

Also provided herein is a method for treating, preventing, or ameliorating a polycystic kidney disease in a subject, comprising administering to the subject a therapeutically effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

Furthermore, provided herein is a method for treating, preventing, or ameliorating a polycystic liver disease in a subject, comprising administering to the subject a therapeutically effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

Provided herein is a method for inhibiting cystogenesis in an organ, comprising contacting the organ with an effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

Provided herein is a method for inhibiting cystogenesis in a kidney, comprising contacting the kidney with an effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

Provided herein is a method for inhibiting cystogenesis in a liver, comprising contacting the liver with an effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

### BRIEF DESCRIPTION OF THE DRAWINGS

FIG. 1 show cell cycle phase distribution in cystic and normal cholangiocytes. *: p<0.01 and **: p<0.0001.

FIG. 2 shows PCNA expression levels in cystic and normal cholangiocytes.

FIG. 3 shows Cdc25A expression levels in cystic and normal cholangiocytes.

FIG. 4A show images of picrosirius red-stained liver sections of PCK rats untreated or treated with vitamin C, vitamin K₃, or APATONE®.

FIG. 4B show effects of vitamin C, vitamin K₃, and APATONE® on hepatic cystogenesis in PCK rats. NT: non-treated; VC: vitamin C-treated; VK3: vitamin K₃-treated; and VC:CK3: APATONE®-treated.

FIG. 5A show images of picrosirius red-stained kidney sections of PCK rats untreated or treated with vitamin C, vitamin K₃, or APATONE®.

FIG. 5B show effects of vitamin C, vitamin K₃, and APATONE® on renal cystogenesis in PCK rats. NT: non-treated; VC: vitamin C-treated; VK3: vitamin K₃-treated; and VC:CK3: APATONE®-treated.

FIG. 6A show images of picrosirius red-stained liver sections of Pkd2^{WS25/-} mice untreated or treated with vitamin C, vitamin K₃, or APATONE®.

FIG. 6B show effects of vitamin C, vitamin K₃, and APATONE® on hepatic cystogenesis in Pkd2^{WS25/-} mice. NT: non-treated; VC: vitamin C-treated; VK3: vitamin K₃-treated; and VC:CK3: APATONE®-treated.

FIG. 7A show images of picrosirius red-stained kidney sections of Pkd2^{WS25/-} mice untreated or treated with vitamin C, vitamin K₃, or APATONE®.

FIG. 7B show effects of vitamin C, vitamin K₃, and APATONE® on renal cystogenesis in Pkd2^{WS25/-} mice. NT: non-treated; VC: vitamin C-treated; VK3: vitamin K₃-treated; and VC:CK3: APATONE®-treated.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, pharmacology, and others described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition (e.g., a polycystic disease), or one or more of the symptoms associated with the disorder, disease, or condition (e.g., a polycystic disease); or alleviating or eradicating the cause(s) of the disorder, disease, or condition (e.g., a polycystic disease) itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The terms “therapeutically effective amount” and “effective amount” are meant to include the amount of a compound or combination of compounds that, when administered, is sufficient to prevent development of, or alleviate to some extent, one or more of the symptoms of the disorder, disease, or condition (e.g., a polycystic disease) being treated. The term “therapeutically effective amount” or “effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 7th Edition, Rowe et al., Eds., The Pharmaceutical Press and the American Pharmaceutical Association: 2012; Handbook of Pharmaceutical Additives, 3rd Edition, Ash and Ash Eds., Gower Publishing Company: 2007; and Pharmaceutical Preformulation and Formulation, 2nd Edition, Gibson Ed., CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition (e.g., a polycystic disease). As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition (e.g., a polycystic disease).

The term “APATONE®” refers to a pharmaceutical composition comprising L-ascorbate and 1,2,3,4-tetrahydro-2-methyl-1,4-dioxo-2-naphthalenesulfonate. In certain embodiments, the term “APATONE®” refers to a pharmaceutical composition, wherein the weight ratio of L-ascorbate to 1,2,3,4-tetrahydro-2-methyl-1,4-dioxo-2-naphthalenesulfonate is about 100 or about 200.

The term “alkyl” refers to a linear or branched saturated monovalent hydrocarbon radical, wherein the alkyl is optionally substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkenyl” refers to a linear or branched monovalent hydrocarbon radical, which contains one or more, in one embodiment, one, two, three, four, or five, in another embodiment, one, carbon-carbon double bond(s). In certain embodiments, the alkenyl is optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkynyl” refers to a linear or branched monovalent hydrocarbon radical, which contains one or more, in one embodiment, one, two, three, four, or five, in another embodiment, one, carbon-carbon triple bond(s). In certain embodiments, the alkynyl is optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₅)_{,} 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “cycloalkyl” refers to a cyclic saturated or non-aromatic unsaturated, bridged or non-bridged monovalent hydrocarbon radical, which is optionally substituted with one or more substituents Q as described herein. In certain embodiments, the cycloalkyl is a cyclic saturated bridged or non-bridged monovalent hydrocarbon radical, which is optionally substituted with one or more substituents Q as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. In certain embodiments, the term “aryl” refers to a bicyclic or tricyclic carbon ring, where one of the rings is aromatic and the others of which may be saturated, partially unsaturated, or aromatic, for example, dihydronaphthyl, indenyl, indanyl, or tetrahydronaphthyl (tetralinyl). In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 1-phenylethyl, 2-phenylethyl, and 3-phenylpropyl. In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each of which is independently selected from O, S, N, and P, in the ring. A heteroaryl group is bonded to the rest of a molecule through its aromatic ring. Each ring of a heteroaryl group can contain one or two O atoms, one or two S atoms, one to four N atoms, and/or one or two P atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each of which is independently selected from O, S, N, and P; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. A heterocyclyl group is bonded to the rest of a molecule through its non-aromatic ring. In certain embodiments, the heterocyclyl is a monocyclic, bicyclic, tricyclic, or tetracyclic ring system, which may be spiro, fused, or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl is optionally substituted with one or more substituents Q as described herein.

The term “halogen”, “halide” or “halo” refers to fluorine, chlorine, bromine, and/or iodine.

The term “optionally substituted” is intended to mean that a group or substituent, such as an alkyl, alkenyl, alkynyl, cycloalkyl, aryl, aralkyl, heteroaryl, and heterocyclyl group, may be substituted with one or more substituents Q, each of which is independently selected from, e.g., (a) oxo (═O), halo, cyano (—CN), and nitro (—NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, four, or five, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{a}R^{d}, —P(O)(OR^{a})R^{d}, —P(O)(OR^{a})(OR^{d}), —SR^{a}, —S(O)R^{a}, —S(O)₂R^{a}, —S(O)NR^{b}R^{c}, and —S(O)₂NR^{b}R^{c}, wherein each R^{a}, R^{b}, R^{e}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heteroaryl or heterocyclyl, each of which is optionally substituted with one or more, in one embodiment, one, two, three, or four, substituents Q^{a}. As used herein, all groups described herein that can be substituted are “optionally substituted,” unless otherwise specified.

In one embodiment, each substituent Q^{a} is independently selected from the group consisting of (a) oxo, cyano, halo, and nitro; and (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —P(O)R^{e}R^{h}, —P(O)(OR^{e})R^{h}, —P(O)(OR^{e})(OR^{h}), —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) R^{f} and R^{g} together with the N atom to which they are attached form heteroaryl or heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less desired enantiomer based on the total weight of the two enantiomers in question.

In describing an optically active compound, the prefixes R and S are used to denote the absolute configuration of the optically active compound about its chiral center(s). The (+) and (−) are used to denote the optical rotation of an optically active compound, that is, the direction in which a plane of polarized light is rotated by the optically active compound. The (−) prefix indicates that an optically active compound is levorotatory, that is, the compound rotates the plane of polarized light to the left or counterclockwise. The (+) prefix indicates that an optically active compound is dextrorotatory, that is, the compound rotates the plane of polarized light to the right or clockwise. However, the sign of optical rotation, (+) and (−), is not related to the absolute configuration of a compound, R and S.

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “chromium-free” refers to a chemical (e.g., a compound or composition) that contains no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. The chromium content can be determined using a conventional technique well known to one of ordinary skill in the art, e.g., inductively coupled plasma (ICP) technique.

The phrase “a single enantiomer, a mixture of enantiomers, or a mixture of diastereomers thereof, or a pharmaceutically acceptable salt, solvate, or hydrate thereof” has the same meaning as the phrase “(i) a single enantiomer, a mixture of enantiomers, or a mixture of diastereomers of the compound referenced therein; (ii) a pharmaceutically acceptable salt, solvate, or hydrate of the compound referenced therein; or (iii) a pharmaceutically acceptable salt, solvate, or hydrate of a single enantiomer, a mixture of enantiomers, or a mixture of diastereomers of the compound referenced therein.”

### Vitamin C

As used herein, the term “vitamin C” refers to L-ascorbic acid or a pharmaceutically acceptable salt thereof; or a pharmaceutically acceptable solvate or hydrate thereof. Vitamin C is also known as L-xyloascorbic acid, 3-oxo-L-gulofuranolactone (enol form), L-3-ketothreohexuronic acid lactone, antiscorbutic vitamin, cevitamic acid, adenex, allercorb, ascorin, ascorteal, ascorvit, cantan, cantaxin, catavin C, cebicure, cebion, cecon, cegiolan, celaskon, celin, cenetone, cereon, cergona, cescorbat, cetamid, cetabe, cetemican, cevalin, cevatine, cevex, cevimin, ce-yl-sol, cevitan, cevitex, cewin, ciamin, cipca, concemin, C-vin, daviamon C, duoscorb, hybrin, laroscorbine, lemascorb, planavit C, proscorbin, redoxon, ribena, scorbacid, scorbu-C, testascorbic, vicelat, vitacee, vitacimin, vitacin, vitascorbol, and xitix.

In one embodiment, vitamin C provided herein is L-ascorbic acid. In another embodiment, vitamin C provided herein is a pharmaceutically acceptable salt of L-ascorbic acid, or a pharmaceutically acceptable solvate or hydrate thereof.

Suitable bases for use in the preparation of pharmaceutically acceptable salts of vitamin C, include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In one embodiment, vitamin C provided herein is an alkali or alkaline earth metal salt of L-ascorbic acid, or a pharmaceutically acceptable solvate or hydrate thereof. In another embodiment, vitamin C provided herein is sodium, potassium, calcium, or magnesium L-ascorbate, or a pharmaceutically acceptable solvate or hydrate thereof. In yet another embodiment, vitamin C provided herein is sodium L-ascorbate, or a pharmaceutically acceptable solvate or hydrate thereof. In yet another embodiment, vitamin C provided herein is sodium L-ascorbate, which is also known as vitamin C sodium, ascorbin, sodascorbate, natrascorb, cenolate, ascorbicin, or cebitate. In yet another embodiment, vitamin C provided herein is potassium L-ascorbate, or a pharmaceutically acceptable solvate or hydrate thereof. In yet another embodiment, vitamin C provided herein is magnesium L-ascorbate, or a pharmaceutically acceptable solvate or hydrate thereof. In yet another embodiment, vitamin C provided herein is magnesium L-ascorbate. In still another embodiment, vitamin C provided herein is zinc L-ascorbate.

In certain embodiments, the vitamin C provided herein is D-ascorbic acid or a pharmaceutically acceptable salt, or a pharmaceutically acceptable solvate or hydrate thereof.

In certain embodiments, the vitamin C provided herein is chromium-free. In certain embodiments, the chromium-free vitamin C provided herein contains no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. In certain embodiments, the chromium-free vitamin C provided herein contains no greater than about 50 ppm chromium. In certain embodiments, the chromium-free vitamin C provided herein contains no greater than about 20 ppm chromium. In certain embodiments, the chromium-free vitamin C provided herein contains no greater than about 10 ppm chromium. In certain embodiments, the chromium-free vitamin C provided herein contains no greater than about 5 ppm chromium. In certain embodiments, the chromium-free vitamin C provided herein contains no greater than about 2 ppm chromium. In certain embodiments, the chromium-free vitamin C provided herein contains no greater than about 1 ppm chromium.

### Vitamin K

As used herein, the term “vitamin K” refers to a 2-methyl-1,4-naphthoquinone of Formula I or II:

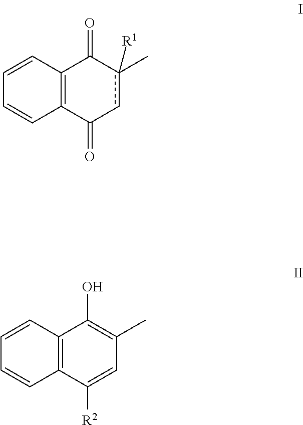

or an enantiomer, a mixture of enantiomers, or a mixture of two or more diastereomers thereof; or a pharmaceutically acceptable salt, solvate, or hydrate thereof; wherein R¹ is alkyl, alkenyl, alkynyl, or —SO₃H; and R₂ is hydroxyl (—OH) or amino (—NH₂).

In certain embodiments, the vitamin K provided herein is vitamin K₁, vitamin K₂, vitamin K₃, vitamin K₄, or vitamin K₅, or a mixture thereof.

In one embodiment, the vitamin K provided herein is vitamin K₁, or a pharmaceutically acceptable salt, solvate, or hydrate thereof. Vitamin K₁ is also known as phylloquinone, [R—[R*₅R*-(E)]]-2-methyl-3-(3,7,11,15-tetramethyl-2-hexadecenyl)-1,4-naphthalenedione, 2-methyl-3-phytyl-1,4-naphthoquinone, 3-phytylmenadione, phytomenadione, phytonadione, aqua-merphyton, konakion, mephyton, mono-day, veda-K₁, and veta-K₁.

In another embodiment, the vitamin K provided herein is vitamin K₂, or a pharmaceutically acceptable salt, solvate, or hydrate thereof. Vitamin K₂ is also known as menaquinones, and 2-methyl-3-all-trans-polyprenyl-1,4-naphthoquinones. Some non-limiting examples of vitamin K₂ include menaquinone 4, which is also known as vitamin K₂₍₂₀₎; menaquinone 6, which is also known as vitamin K₂₍₃₀₎; and menaquinone 7, which is also known as vitamin K₂₍₃₅₎.

In yet another embodiment, the vitamin K provided herein is vitamin K₃, or a pharmaceutically acceptable salt, solvate, or hydrate thereof. Vitamin K₃ is also known as menadione, 2-methyl-1,4-naphthalenedione, 2-methyl-1,4-naphthoquinone, menaphthone, vitamin K₂₍₀₎, kanone, kappaxin, kayklot, kayquinone, klottone, kolklot, and thyloquinone, 1,2,3,4-tetrahydro-2-methyl-1,4-dioxo-2-naphthalenesulfonic acid, and sodium 1,2,3,4-tetrahydro-2-methyl-1,4-dioxo-2-naphthalenesulfonate.

In one embodiment, the vitamin K provided herein is 1,2,3,4-tetrahydro-2-methyl-1,4-dioxo-2-naphthalenesulfonic acid, or a pharmaceutically acceptable salt, solvate, or hydrate thereof. In another embodiment, the vitamin K provided herein is 1,2,3,4-tetrahydro-2-methyl-1,4-dioxo-2-naphthalenesulfonate (also known as menadione bisulfite), or a pharmaceutically acceptable solvate or hydrate thereof.

Suitable bases for use in the preparation of pharmaceutically acceptable salts of vitamin K, include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In one embodiment, vitamin K₃ provided herein is an alkali or alkaline earth metal salt of 1,2,3,4-tetrahydro-2-methyl-1,4-dioxo-2-naphthalenesulfonic acid, or a pharmaceutically acceptable solvate or hydrate thereof. In another embodiment, vitamin K₃ provided herein is sodium, potassium, calcium, or magnesium 1,2,3,4-tetrahydro-2-methyl-1,4-dioxo-2-naphthalenesulfonate, or a pharmaceutically acceptable solvate or hydrate thereof. In yet another embodiment, vitamin K₃ provided herein is sodium 1,2,3,4-tetrahydro-2-methyl-1,4-dioxo-2-naphthalenesulfonate, or a pharmaceutically acceptable solvate or hydrate thereof. In yet another embodiment, vitamin K₃ provided herein is potassium 1,2,3,4-tetrahydro-2-methyl-1,4-dioxo-2-naphthalenesulfonate, or a pharmaceutically acceptable solvate or hydrate thereof. In yet another embodiment, vitamin K₃ provided herein is magnesium 1,2,3,4-tetrahydro-2-methyl-1,4-dioxo-2-naphthalenesulfonate, or a pharmaceutically acceptable solvate or hydrate thereof. In yet another embodiment, vitamin K₃ provided herein is sodium 1,2,3,4-tetrahydro-2-methyl-1,4-dioxo-2-naphthalenesulfonate. In yet another embodiment, vitamin K₃ provided herein is anhydrous sodium 1,2,3,4-tetrahydro-2-methyl-1,4-dioxo-2-naphthalenesulfonate. In yet another embodiment, vitamin K₃ provided herein is sodium 1,2,3,4-tetrahydro-2-methyl-1,4-dioxo-2-naphthalenesulfonate hydrate. In still another embodiment, vitamin K₃ provided herein is sodium 1,2,3,4-tetrahydro-2-methyl-1,4-dioxo-2-naphthalenesulfonate trihydrate.

In certain embodiments, the vitamin K provided herein is vitamin K₄, or a pharmaceutically acceptable salt, solvate, or hydrate thereof. Vitamin K₄ is also known as menadiol, 2-methyl-1,4-naphthalenediol, 2-methyl-1,4-naphthohydroquinone, 2-methyl-1,4-naphthoquinol, and dihydrovitamin K₃.

In certain embodiments, the vitamin K provided herein comprises vitamin K₃ and vitamin K₄, or pharmaceutically acceptable salts, solvates, or hydrates thereof.

In certain embodiments, the vitamin K provided herein is vitamin K₅, or a pharmaceutically acceptable salt, solvate, or hydrate thereof. Vitamin K₅ is also known as 4-amino-2-methyl-1-naphthalenol, 4-amino-2-methyl-1-naphthol, 1-hydroxy-2-methyl-4-aminonaphalene, 2-methyl-4-amino-1-hydroxynaphthalene, 2-methyl-4-amino-1-naphthol, 3-methyl-4-hydroxy-1-naphthylamine, and synkamin.

In certain embodiments, the vitamin K provided herein is chromium-free. In certain embodiments, the chromium-free vitamin K provided herein contains no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. In certain embodiments, the chromium-free vitamin K provided herein contains no greater than about 50 ppm chromium. In certain embodiments, the chromium-free vitamin K provided herein contains no greater than about 20 ppm chromium. In certain embodiments, the chromium-free vitamin K provided herein contains no greater than about 10 ppm chromium. In certain embodiments, the chromium-free vitamin K provided herein contains no greater than about 5 ppm chromium. In certain embodiments, the chromium-free vitamin K provided herein contains no greater than about 2 ppm chromium. In certain embodiments, the chromium-free vitamin K provided herein contains no greater than about 1 ppm chromium.

In certain embodiments, the vitamin K provided herein is chromium-free vitamin K₃. In certain embodiments, the chromium-free vitamin K₃ provided herein contains no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. In certain embodiments, the chromium-free vitamin K₃ provided herein contains no greater than about 50 ppm chromium. In certain embodiments, the chromium-free vitamin K₃ provided herein contains no greater than about 20 ppm chromium. In certain embodiments, the chromium-free vitamin K₃ provided herein contains no greater than about 10 ppm chromium. In certain embodiments, the chromium-free vitamin K₃ provided herein contains no greater than about 5 ppm chromium. In certain embodiments, the chromium-free vitamin K₃ provided herein contains no greater than about 2 ppm chromium. In certain embodiments, the chromium-free vitamin K₃ provided herein contains no greater than about 1 ppm chromium.

In certain embodiments, the vitamin K provided herein is chromium-free sodium 1,2,3,4-tetrahydro-2-methyl-1,4-dioxo-2-naphthalenesulfonate. In certain embodiments, the chromium-free sodium 1,2,3,4-tetrahydro-2-methyl-1,4-dioxo-2-naphthalenesulfonate contains no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. In certain embodiments, the chromium-free sodium 1,2,3,4-tetrahydro-2-methyl-1,4-dioxo-2-naphthalenesulfonate contains no greater than about 50 ppm chromium. In certain embodiments, the chromium-free sodium 1,2,3,4-tetrahydro-2-methyl-1,4-dioxo-2-naphthalenesulfonate contains no greater than about 20 ppm chromium. In certain embodiments, the chromium-free sodium 1,2,3,4-tetrahydro-2-methyl-1,4-dioxo-2-naphthalenesulfonate contains no greater than about 10 ppm chromium. In certain embodiments, the chromium-free sodium 1,2,3,4-tetrahydro-2-methyl-1,4-dioxo-2-naphthalenesulfonate contains no greater than about 5 ppm chromium. In certain embodiments, the chromium-free sodium 1,2,3,4-tetrahydro-2-methyl-1,4-dioxo-2-naphthalenesulfonate contains no greater than about 2 ppm chromium. In certain embodiments, the chromium-free sodium 1,2,3,4-tetrahydro-2-methyl-1,4-dioxo-2-naphthalenesulfonate contains no greater than about 1 ppm chromium.

In certain embodiments, the chromium-free vitamin K₃ provided herein is made via a cerium mediated electrochemical technology (CETECH™) as described in U.S. Pat. No. 6,468,414, the disclosure of which is incorporated herein by reference in its entirety. Alternatively, chromium-free vitamin K₃ is available from commercial sources, such as PRO-K™ (Lonza Group Ltd, Switzerland).

### Pharmaceutical Compositions

In one embodiment, provided herein is a pharmaceutical composition comprising vitamin C, or a pharmaceutically acceptable salt, solvate, or hydrate thereof; vitamin K, or a single enantiomer, a mixture of enantiomers, or a mixture of diastereomers thereof, or a pharmaceutically acceptable salt, solvate, or hydrate thereof; and optionally a pharmaceutically acceptable excipient.

In another embodiment, provided herein is a pharmaceutical composition comprising vitamin C, or a pharmaceutically acceptable salt, solvate, or hydrate thereof; chromium-free vitamin K, or a single enantiomer, a mixture of enantiomers, or a mixture of diastereomers thereof, or a pharmaceutically acceptable salt, solvate, or hydrate thereof; and optionally a pharmaceutically acceptable excipient.

In yet another embodiment, provided herein is a pharmaceutical composition comprising chromium-free vitamin C, or a pharmaceutically acceptable salt, solvate, or hydrate thereof; chromium-free vitamin K, or a single enantiomer, a mixture of enantiomers, or a mixture of diastereomers thereof, or a pharmaceutically acceptable salt, solvate, or hydrate thereof; and optionally a pharmaceutically acceptable excipient.

In still another embodiment, provided herein is a chromium-free pharmaceutical composition comprising vitamin C, or a pharmaceutically acceptable salt, solvate, or hydrate thereof; vitamin K, or a single enantiomer, a mixture of enantiomers, or a mixture of diastereomers thereof, or a pharmaceutically acceptable salt, solvate, or hydrate thereof; and optionally a pharmaceutically acceptable excipient.

In one embodiment, the weight ratio of vitamin C to vitamin K in the pharmaceutical compositions provided herein is ranging from about 1 to about 500, from about 4 to about 500, from about 10 to about 500, from about 50 to about 500, from about 25 to about 250, or from about 50 to about 200, from about 50 to about 150, or from about 80 to about 120. In another embodiment, the weight ratio of vitamin C to vitamin K in the pharmaceutical compositions provided herein is about 1, about 2, about 4, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250. In yet another embodiment, the weight ratio of vitamin C to vitamin K in the pharmaceutical compositions provided herein is about 100. In still another embodiment, the weight ratio of vitamin C to vitamin K in the pharmaceutical compositions provided herein is about 200.

In certain embodiments, the pharmaceutical compositions provided herein are chromium-free. In certain embodiments, the pharmaceutical compositions provided herein contain no more than about 100 ppm, no more than about 50 ppm, no more than about 20 ppm, no more than about 10 ppm, no more than about 5 ppm, no more than about 2 ppm, no more than about 1 ppm, no more than about 0.1 ppm, no more than about 10 ppb, or no more than about 1 ppb of chromium. In certain embodiments, the pharmaceutical compositions provided herein contain no greater than about 10 ppm chromium. In certain embodiments, the pharmaceutical compositions provided herein contain no greater than about 5 ppm chromium. In certain embodiments, the pharmaceutical compositions provided herein contain no greater than about 2 ppm chromium. In certain embodiments, the pharmaceutical compositions provided herein contain no greater than about 1 ppm chromium.

The pharmaceutical compositions provided herein may be formulated in various dosage forms for oral, parenteral, and topical administration. The pharmaceutical compositions may also be formulated as modified release dosage forms, including delayed-, extended-, prolonged-, sustained-, pulsatile-, controlled-, accelerated-, fast-, targeted-, programmed-release, time-release, thermal-release, pH dependent release, biodegradation-release, and gastric retention dosage forms. These dosage forms can be prepared according to conventional methods and techniques known to those skilled in the art (See, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2003; Vol. 126).

In one embodiment, the pharmaceutical compositions provided herein are formulated in a dosage form for oral administration. In another embodiment, the pharmaceutical compositions provided herein are formulated in a dosage form for parenteral administration. In yet another embodiment, the pharmaceutical compositions provided herein are formulated in a dosage form for intravenous administration. In yet another embodiment, the pharmaceutical compositions provided herein are formulated in a dosage form for topical administration. In still another embodiment, the pharmaceutical compositions provided herein are formulated in a dosage form for local injection.

In one embodiment, the pharmaceutical compositions provided herein are formulated together as a capsule. In one embodiment, the capsule contains from about 10 mg to about 1,000 mg, from about 25 mg to about 900 mg, from about 50 mg to about 800 mg, from about 100 mg to about 700 mg, from about 200 mg to about 600 mg, from about 300 mg to about 600 mg, or from about 400 mg to about 600 mg of vitamin C, or a pharmaceutically acceptable salt, solvate, or hydrate thereof; and from about 0.1 mg to about 10 mg, from about 1 mg to about 9 mg, from about 2 mg to about 8 mg, from about 3 mg to about 7 mg, or from about 4 mg to about 6 mg of vitamin K, or a single enantiomer, a mixture of enantiomers, or a mixture of diastereomers thereof, or a pharmaceutically acceptable salt, solvate, or hydrate thereof. In another embodiment, the capsule contains from about 400 mg to about 600 mg of vitamin C, or a pharmaceutically acceptable salt, solvate, or hydrate thereof and from about 4 mg to about 6 mg of vitamin K, or a single enantiomer, a mixture of enantiomers, or a mixture of diastereomers thereof, or a pharmaceutically acceptable salt, solvate, or hydrate thereof. In yet another embodiment, the capsule contains about 200 mg, about 300 mg, about 400, about 500, about 600 mg, about 700 mg, about 800 mg, or about 900 mg of vitamin C, or a pharmaceutically acceptable salt, solvate, or hydrate thereof and about 1 mg, about 2 mg, about 3 mg, about 4 mg, about 5 mg, about 6 mg, about 7 mg, about 8 mg, about 9 mg, or about 10 mg of vitamin K, or a single enantiomer, a mixture of enantiomers, or a mixture of diastereomers thereof, or a pharmaceutically acceptable salt, solvate, or hydrate thereof. In still another embodiment, the capsule contains about 500 mg of vitamin C, or a pharmaceutically acceptable salt, solvate, or hydrate thereof and about 5 mg of vitamin K, or a single enantiomer, a mixture of enantiomers, or a mixture of diastereomers thereof, or a pharmaceutically acceptable salt, solvate, or hydrate thereof. In certain embodiments, the capsule consists essentially of vitamins C, or a pharmaceutically acceptable salt, solvate, or hydrate thereof and K, or a single enantiomer, a mixture of enantiomers, or a mixture of diastereomers thereof, or a pharmaceutically acceptable salt, solvate, or hydrate thereof.

In one embodiment, vitamin C in the pharmaceutical compositions provided herein is L-ascorbic acid or a pharmaceutically acceptable salt thereof, or a pharmaceutically acceptable solvate or hydrate thereof. In another embodiment, vitamin C in the pharmaceutical compositions provided herein is an alkali or alkaline earth metal salt of L-ascorbic acid, or a pharmaceutically acceptable solvate or hydrate thereof. In yet another embodiment, vitamin C in the pharmaceutical compositions provided herein is sodium, potassium, calcium, or magnesium salt of L-ascorbic acid, or a pharmaceutically acceptable solvate or hydrate thereof. In yet another embodiment, vitamin C in the pharmaceutical compositions provided herein is sodium L-ascorbate. In still another embodiment, vitamin C in the pharmaceutical compositions provided herein is magnesium L-ascorbate.

In one embodiment, vitamin K in the pharmaceutical compositions provided herein is vitamin K₃, or a pharmaceutically acceptable salt, solvate, or hydrate thereof. In another embodiment, vitamin K in the pharmaceutical compositions provided herein is 1,2,3,4-tetrahydro-2-methyl-1,4-dioxo-2-naphthalenesulfonic acid, or a pharmaceutically acceptable salt, solvate, or hydrate thereof. In yet another embodiment, vitamin K in the pharmaceutical compositions provided herein is an alkali or alkaline earth metal salt of 1,2,3,4-tetrahydro-2-methyl-1,4-dioxo-2-naphthalenesulfonic acid, or a pharmaceutically acceptable solvate or hydrate thereof. In yet another embodiment, vitamin K in the pharmaceutical compositions provided herein is sodium, potassium, calcium, or magnesium 1,2,3,4-tetrahydro-2-methyl-1,4-dioxo-2-naphthalenesulfonate, or a pharmaceutically acceptable solvate or hydrate thereof. In yet another embodiment, vitamin K in the pharmaceutical compositions provided herein is sodium 1,2,3,4-tetrahydro-2-methyl-1,4-dioxo-2-naphthalenesulfonate, or a pharmaceutically acceptable solvate or hydrate thereof. In yet another embodiment, vitamin K in the pharmaceutical compositions provided herein is potassium 1,2,3,4-tetrahydro-2-methyl-1,4-dioxo-2-naphthalenesulfonate, or a pharmaceutically acceptable solvate or hydrate thereof. In yet another embodiment, vitamin K in the pharmaceutical compositions provided herein is magnesium 1,2,3,4-tetrahydro-2-methyl-1,4-dioxo-2-naphthalenesulfonate, or a pharmaceutically acceptable solvate or hydrate thereof. In yet another embodiment, vitamin K in the pharmaceutical compositions provided herein is sodium 1,2,3,4-tetrahydro-2-methyl-1,4-dioxo-2-naphthalenesulfonate. In yet another embodiment, vitamin K in the pharmaceutical compositions provided herein is anhydrous sodium 1,2,3,4-tetrahydro-2-methyl-1,4-dioxo-2-naphthalenesulfonate. In yet another embodiment, vitamin K in the pharmaceutical compositions provided herein is sodium 1,2,3,4-tetrahydro-2-methyl-1,4-dioxo-2-naphthalenesulfonate hydrate. In still another embodiment, vitamin K in the pharmaceutical compositions provided herein is sodium 1,2,3,4-tetrahydro-2-methyl-1,4-dioxo-2-naphthalenesulfonate trihydrate.

In one embodiment, the capsule contains about 500 mg of sodium L-ascorbate, and about 5 mg of sodium 1,2,3,4-tetrahydro-2-methyl-1,4-dioxo-2-naphthalenesulfonate or a hydrate thereof. In another embodiment, the capsule contains about 500 mg of magnesium L-ascorbate, and about 5 mg of sodium 1,2,3,4-tetrahydro-2-methyl-1,4-dioxo-2-naphthalenesulfonate or hydrate thereof. In yet another embodiment, the capsule contains about 500 mg of sodium L-ascorbate and about 5 mg of anhydrous sodium 1,2,3,4-tetrahydro-2-methyl-1,4-dioxo-2-naphthalenesulfonate. In yet another embodiment, the capsule contains about 500 mg of sodium L-ascorbate and about 5 mg of sodium 1,2,3,4-tetrahydro-2-methyl-1,4-dioxo-2-naphthalenesulfonate trihydrate. In yet another embodiment, the capsule contains about 500 mg of magnesium L-ascorbate and about 5 mg of anhydrous sodium 1,2,3,4-tetrahydro-2-methyl-1,4-dioxo-2-naphthalenesulfonate. In still another embodiment, the capsule contains about 500 mg of magnesium L-ascorbate and about 5 mg of sodium 1,2,3,4-tetrahydro-2-methyl-1,4-dioxo-2-naphthalenesulfonate trihydrate.

In one embodiment, the capsule consists essentially of vitamin C, or a pharmaceutically acceptable salt, solvate, or hydrate thereof, in combination with vitamin K, or a single enantiomer, a mixture of enantiomers, or a mixture of diastereomers thereof, or a pharmaceutically acceptable salt, solvate, or hydrate thereof. In certain embodiments, the capsule consists essentially of vitamin C, or a pharmaceutically acceptable salt, solvate, or hydrate thereof, in combination with vitamin K₃, or a pharmaceutically acceptable salt, solvate, or hydrate thereof. In one embodiment, the capsule consists essentially of sodium L-ascorbate, and sodium 1,2,3,4-tetrahydro-2-methyl-1,4-dioxo-2-naphthalenesulfonate or a hydrate thereof. In another embodiment, the capsule consists essentially of magnesium L-ascorbate, and sodium 1,2,3,4-tetrahydro-2-methyl-1,4-dioxo-2-naphthalenesulfonate or hydrate thereof. In yet another embodiment, the capsule consists essentially of sodium L-ascorbate and anhydrous sodium 1,2,3,4-tetrahydro-2-methyl-1,4-dioxo-2-naphthalenesulfonate. In yet another embodiment, the capsule consists essentially of sodium L-ascorbate and sodium 1,2,3,4-tetrahydro-2-methyl-1,4-dioxo-2-naphthalenesulfonate trihydrate. In yet another embodiment, the capsule consists essentially of magnesium L-ascorbate and anhydrous sodium 1,2,3,4-tetrahydro-2-methyl-1,4-dioxo-2-naphthalenesulfonate. In still another embodiment, the capsule consists essentially of magnesium L-ascorbate and sodium 1,2,3,4-tetrahydro-2-methyl-1,4-dioxo-2-naphthalenesulfonate trihydrate.

The pharmaceutical compositions provided herein can also be formulated as known to those skilled in the art. Some examples of vitamins C and K containing pharmaceutical compositions are described in U.S. Pat. No. 7,091,241, the disclosure of which is incorporated herein by reference in its entirety.

The pharmaceutical compositions provided herein may be provided in a unit-dosage or multiple-dosage form. A unit-dosage form, as used herein, refers to physically discrete a unit suitable for administration to a subject, e.g., a human and animal subject, and packaged individually as is known in the art. Each unit-dose contains a predetermined quantity of an active ingredient(s) sufficient to produce the desired therapeutic effect, in association with the required pharmaceutical excipients. Examples of a unit-dosage form include an ampoule, syringe, and individually packaged tablet, capsule, and topical gel. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, phosphoric acid, sodium bicarbonate, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is (are) dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as hydrogen peroxide, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein is a method for treating, preventing, or ameliorating a polycystic disease in a subject, comprising administering to the subject vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

In certain embodiments, the polycystic disease is a polycystic kidney disease, a polycystic liver disease, a polycystic pancreas disease, a polycystic ovarian syndrome, or a combination thereof.

In certain embodiments, the polycystic disease is a polycystic kidney disease. In certain embodiments, the polycystic kidney disease is an autosomal dominant polycystic kidney disease (ADPKD) or an autosomal recessive polycystic kidney disease (ARPKD).

In certain embodiments, the polycystic kidney disease is ADPKD. In certain embodiments, ADPKD is caused by one or more mutations in PKD1, PKD2, and/or PKD3. In certain embodiments, ADPKD is caused by a mutation in PKD1. In certain embodiments, ADPKD is caused by a mutation in PKD2. In certain embodiments, ADPKD is caused by a mutation in PKD3.

In certain embodiments, the polycystic kidney disease is ARPKD. In certain embodiments, ARPKD is caused by one or more mutations in PKHD1. In certain embodiments, ARPKD is caused by a mutation in PKHD1.

In certain embodiments, the polycystic disease is a polycystic liver disease (PLD). In certain embodiments, the PLD is an isolated PLD. In certain embodiments, the PLD is an autosomal dominant polycystic liver disease (ADPLD). In certain embodiments, the PLD is an isolated ADPLD. In certain embodiments, the PLD is caused by one or more mutations in PPRKCSH and/or SEC63. In certain embodiments, the PLD is caused by a mutation in PPRKCSH. In certain embodiments, the PLD is caused by a mutation in SEC63. In certain embodiments, the PLD is associated with ARPKD. In certain embodiments, the PLD is associated with ADPKD.

In certain embodiments, the polycystic disease is a polycystic kidney disease and a polycystic liver disease.

In certain embodiments, the polycystic disease is a polycystic pancreas disease. In certain embodiments, the polycystic disease is a polycystic ovarian syndrome.

In another embodiment, provided herein is a method for treating, preventing, or ameliorating a polycystic kidney disease in a subject, comprising administering to the subject vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

In yet another embodiment, provided herein is a method for treating, preventing, or ameliorating a polycystic liver disease in a subject, comprising administering to the subject vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

In certain embodiments, the combination of vitamins C and K has a synergetic effect in treating, preventing, or ameliorating one or more symptoms of a polycystic disease as compared to the administration of either compound alone. In certain embodiments, the combination of sodium or magnesium L-ascorbate and sodium 1,2,3,4-tetrahydro-2-methyl-1,4-dioxo-2-naphtalenesulfonate has a synergetic effect in treating, preventing, or ameliorating one or more symptoms of a polycystic disease as compared to the administration of either compound alone.

Without being limited by any theory, a synergistic effect of the combination of vitamins C and K permits the use of lower dosages of vitamin C and/or K, and/or less frequent administration of the combination to a subject having a polycystic disease. The ability to utilize lower dosages of the combination and/or to administer the combination less frequently reduces the toxicity associated with the administration of the combination to a subject without reducing the efficacy of the combination in treating, preventing, or ameliorating one or more symptoms of a polycystic disease. In addition, a synergistic effect can result in improved efficacy of vitamin C and/or K in treating, preventing, or ameliorating one or more symptoms of a polycystic disease. Furthermore, a synergistic effect of the combination may avoid or reduce adverse or unwanted side effects associated with the use of either vitamin C or K alone.

In certain embodiments, vitamin C, vitamin K as used in the methods provided herein are delivered as a single dose such as, e.g., as a single bolus injection, or as a single oral tablet or pill. In certain embodiments, vitamin C, vitamin K as used in the methods provided herein are administered over time, such as, e.g., continuous infusion over time or divided bolus doses over time.

In certain embodiments, the weight ratio of vitamin C to vitamin K as used in the methods provided herein is ranging from about 1 to about 500, from about 4 to about 500, from about 10 to about 500, from about 50 to about 500, from about 25 to about 250, or from about 50 to about 200, from about 50 to about 150, or from about 80 to about 120. In certain embodiments, the weight ratio of vitamin C to vitamin K as used in the methods provided herein is about 1, about 2, about 4, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250. In certain embodiments, the weight ratio of vitamin C to vitamin K as used in the methods provided herein is about 100. In certain embodiments, the weight ratio of vitamin C to vitamin K as used in the methods provided herein is about 200.

In certain embodiments, vitamins C and K as used in the methods provided herein are administered once daily (QD), or divided into multiple daily doses such as twice daily (BID), three times daily (TID), four times daily (QID), five times daily, six times daily, seven times daily, eight times daily, nine times daily, or ten times daily. In certain embodiments, vitamin C as used in the methods provided herein is administered once daily (QD), or divided into multiple daily doses such as twice daily (BID), three times daily (TID), four times daily (QID), five times daily, six times daily, seven times daily, eight times daily, nine times daily, or ten times daily. In certain embodiments, vitamin K as used in the methods provided herein is administered once daily (QD), or divided into multiple daily doses such as twice daily (BID), three times daily (TID), four times daily (QID), five times daily, six times daily, seven times daily, eight times daily, nine times daily, or ten times daily.

In certain embodiments, vitamin C and/or vitamin K as used in the methods provided herein are administered from about 1 to about 20 times a day, from about 1 to about 15 times a day, from about 1 to about 10 times a day, or from about 1 to about 5 times a day. In certain embodiments, vitamin C and/or vitamin K as used in the methods provided herein are administered every 1 to 10 hour(s), every 2 to 8 hours, every 3 to 7 hours, every 4 to 6 hours, or every 5 to 6 hours. In certain embodiments, vitamin C and/or vitamin K as used in the methods provided herein are administered every hour, every 2 hours, every 3 hours, every 4 hours, every 5 hours, every 6 hours, every 7 hours, every 8 hours, every 9 hours, or every 10 hours. In certain embodiments, vitamin C and/or vitamin K as used in the methods provided herein are administered once a day. In certain embodiments, vitamin C and/or vitamin K as used in the methods provided herein are administered 5 times a day. In certain embodiments, vitamin C and/or vitamin K as used in the methods provided herein are administered 10 times a day. In certain embodiments, vitamin C and/or vitamin K as used in the methods provided herein are administered every 4, 5, or 6 hours. In certain embodiments, vitamins C and K are administered daily.

In certain embodiments, vitamin C as used in the methods provided herein is administered to the subject in an amount ranging from about 1 to about 1,000 mg/kg/day, from about 5 to about 500 mg/kg/day, or from about 10 to about 100 mg/kg/day. In certain embodiments, vitamin C as used in the methods provided herein is administered to the subject in an amount of about 10 mg/kg/day, about 20 mg/kg/day, about 30 mg/kg/day, about 40 mg/kg/day, about 50 mg/kg/day, about 60 mg/kg/day, about 70 mg/kg/day, about 80 mg/kg/day, about 90 mg/kg/day, about 100 mg/kg/day, about 200 mg/kg/day, about 300 mg/kg/day, about 400 mg/kg/day, or about 500 mg/kg/day.

In certain embodiments, vitamin K as used in the methods provided herein is administered to the subject in an amount ranging from about 0.01 to about 50 mg/kg/day, from about 0.015 to about 50 mg/kg/day, from about 0.05 to about 40 mg/kg/day, from about 0.2 to about 30 mg/kg/day, or from about 10 to about 30 mg/kg/day. In certain embodiments, vitamin K as used in the methods provided herein is administered to the subject in an amount of about 0.015 mg/kg/day, about 5 mg/kg/day, about 25 mg/kg/day, or about 30 mg/kg/day.

The administered dose of vitamins C and vitamin K can also be expressed in units other than the unit “mg/kg/day” or “g/kg/day.” For example, doses for parenteral administration can be expressed as mg/m²/day. One of ordinary skill in the art would readily know how to convert doses from mg/kg/day to mg/m²/day, given either the height or weight of a subject or both.

In certain embodiments, vitamin C as used in the methods provided herein is administered to the subject in an amount ranging from about 0.1 g to about 3 g every four hours. In certain embodiments, vitamin K as used in the methods provided herein is administered to the subject in an amount ranging from about 0.2 mg to about 300 mg every four hours.

In certain embodiments, vitamin C as used in the methods provided herein is administered to the subject in an amount ranging from about 500 mg to about 3,000 mg a day. In certain embodiments, vitamin K as used in the methods provided herein is administered to the subject in an amount ranging from about 3 mg to about 30 mg a day. In certain embodiments, vitamin C as used in the methods provided herein is administered to the subject in an amount ranging from about 500 mg to about 10,000 mg a day. In certain embodiments, vitamin K as used in the methods provided herein is administered to the subject in an amount ranging from about 3 mg to about 100 mg a day. In certain embodiments, vitamin C as used in the methods provided herein is administered to the subject in an amount of greater than about 500 mg a day. In certain embodiments, vitamin K as used in the methods provided herein is administered to the subject in an amount of greater than about 3 mg a day. In certain embodiments, vitamin C as used in the methods provided herein is administered to the subject in an amount up to about 10,000 mg a day. In certain embodiments, vitamin K as used in the methods provided herein is administered to the subject in an amount up to about 100 mg a day. In certain embodiments, vitamin C as used in the methods provided herein is administered to the subject in an amount up to about 20,000 mg a day. In certain embodiments, vitamin K as used in the methods provided herein is administered to the subject in an amount up to about 200 mg a day. In certain embodiments, vitamin C as used in the methods provided herein is administered to the subject in an amount up to about 30,000 mg a day. In certain embodiments, vitamin K as used in the methods provided herein is administered to the subject in an amount up to about 300 mg a day. In certain embodiments, vitamin C as used in the methods provided herein is administered to the subject in an amount up to about 40,000 mg a day. In certain embodiments, vitamin K as used in the methods provided herein is administered to the subject in an amount up to about 400 mg a day. In certain embodiments, vitamin C as used in the methods provided herein is administered to the subject in an amount up to about 50,000 mg a day. In certain embodiments, vitamin K as used in the methods provided herein is administered to the subject in an amount up to about 500 mg a day. In certain embodiments, vitamin C as used in the methods provided herein is administered to the subject in an amount up to about 60,000 mg a day. In certain embodiments, vitamin K as used in the methods provided herein is administered to the subject in an amount up to about 600 mg a day. In certain embodiments, vitamin C as used in the methods provided herein is administered to the subject in an amount up to about 70,000 mg a day. In certain embodiments, vitamin K as used in the methods provided herein is administered to the subject in an amount up to about 700 mg a day. In certain embodiments, vitamin C as used in the methods provided herein is administered to the subject in an amount up to about 80,000 mg a day. In certain embodiments, vitamin K as used in the methods provided herein is administered to the subject in an amount up to about 800 mg a day. In certain embodiments, vitamin C as used in the methods provided herein is administered to the subject in an amount up to about 90,000 mg a day. In certain embodiments, vitamin K as used in the methods provided herein is administered to the subject in an amount up to about 900 mg a day. In certain embodiments, vitamin C as used in the methods provided herein is administered to the subject in an amount up to about 100,000 mg a day. In certain embodiments, vitamin K as used in the methods provided herein is administered to the subject in an amount up to about 1,000 mg a day. In certain embodiments, vitamin C as used in the methods provided herein is administered to the subject in an amount up to about 200,000 mg a day. In certain embodiments, vitamin K as used in the methods provided herein is administered to the subject in an amount up to about 2,000 mg a day.

In certain embodiments, vitamin C as used in the methods provided herein is administered to the subject in an amount ranging from about 2,000 mg to about 3,000 mg a day; and vitamin K is administered to the subject in an amount ranging from about 12 mg to about 19 mg a day. In certain embodiments, vitamin C as used in the methods provided herein is administered to the subject in an amount ranging from about 2,000 mg to about 3,000 mg a day; and vitamin K is administered to the subject in an amount ranging from about 20 mg to about 30 mg a day.

In certain embodiments, vitamin C as used in the methods provided herein is administered to the subject in an amount of about 2,000 mg a day; and vitamin K is administered to the subject in an amount of about 12 mg a day. In certain embodiments, vitamin C as used in the methods provided herein is administered to the subject in an amount of about 3,000 mg a day; and vitamin K is administered to the subject in an amount of about 19 mg a day.

In certain embodiments, vitamin C as used in the methods provided herein is administered to the subject in an amount of about 2,000 mg a day; and vitamin K is administered to the subject in an amount of about 20 mg a day. In certain embodiments, vitamin C as used in the methods provided herein is administered to the subject in an amount of about 3,000 mg a day; and vitamin K is administered to the subject in an amount of about 30 mg a day.

In certain embodiments, vitamins C and K are administered as one or more capsules, each comprising about 500 mg of sodium L-ascorbate and about 3 mg of sodium 1,2,3,4-tetrahydro-2-methyl-1,4-dioxo-2-naphthalenesulfonate. In certain embodiments, vitamins C and K are administered as one or more capsules, each comprising about 500 mg of sodium L-ascorbate and about 5 mg of sodium 1,2,3,4-tetrahydro-2-methyl-1,4-dioxo-2-naphthalenesulfonate.

Depending on the condition of the polycystic disease to be treated and the subject's condition, vitamins C and K used in the methods provided herein can be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) route of administration. In certain embodiments, vitamins C and K used in the methods provided herein are administered by oral, parenteral, intravenous, or topical route of administration. Vitamins C and K used in the methods provided herein may be formulated, alone or together, in suitable dosage unit with one or more pharmaceutically acceptable excipients appropriate for each route of administration.

In one embodiment, vitamin C is administered orally. In another embodiment, vitamin C is administered parenterally. In yet another embodiment, vitamin C is administered intravenously. In still another embodiment, vitamin C is administered topically.

In one embodiment, vitamin K is administered orally. In another embodiment, vitamin K is administered parenterally. In yet another embodiment, vitamin K is administered intravenously. In still another embodiment, vitamin K is administered topically.

The routes of administration of vitamins C and K can be the same or different. In certain embodiments, both vitamins C and K are administered orally.

In one embodiment, vitamin C is administered concurrently with vitamin K. In another embodiment, vitamin C is administered separately with vitamin K. In yet another embodiment, vitamin C is administered sequentially with vitamin K. In yet another embodiment, vitamin C is administered before vitamin K. In yet another embodiment, vitamin C is administered after vitamin K.

In certain embodiments, vitamin C and vitamin K are administered together in a single composition comprising vitamin C, or a pharmaceutically acceptable salt, solvate, or hydrate thereof, and vitamin K, or a single enantiomer, a mixture of enantiomers, or a mixture of diastereomers thereof, or a pharmaceutically acceptable salt, solvate, or hydrate thereof.

In certain embodiments, a combination of vitamin C and vitamin K₃ is administered to the subject after mealtime.

In certain embodiments, the subject is a mammal. In certain embodiments, the mammal is a human.

The methods provided herein encompass treating a subject regardless of patient's age, although some conditions, diseases, or disorders are more common in certain age groups. In certain embodiments, the subject is a male. In certain embodiments, the subject is a female. In certain embodiments, the subject is an elderly.

In certain embodiments, the subject is a human with an age of no less than about 20 years, no less than about 30 years, no less than about 40 years, no less than about 45 years, no less than about 50 years, no less than about 55 years, no less than about 60 years, no less than about 65 years, no less than about 70 years, no less than about 75 years, or no less than about 80 years. In certain embodiments, the subject is a human with an age of above about 60, above about 65, above about 70, or above about 75. In certain embodiments, the subject is a human with an age ranging from about 20 to about 30 years, from about 30 to about 40 years, from about 40 to about 50 years, from about 50 to about 60 years, from about 60 to about 70 years, or from about 70 to about 80 years. In certain embodiments, the subject is a human with an age ranging from about 1 to about 110 years, from about 1 to about 100 years, from about 1 to about 90 years, from about 1 to about 80 years, from about 1 to about 70 years, from about 1 to about 60 years, or from about 1 to about 50 years.

In certain embodiments, the subject is a human with an age of no greater than about 20 years, no greater than about 15, no greater than about 10, no greater than about 5, or no greater than about 2.

In certain embodiments, the subject to be treated with one of the methods provided herein has not been treated with any of the methods provided herein. In certain embodiments, the subject to be treated with one of the methods provided herein has been treated with one of the methods provided herein.

The combination regimen can be administered repetitively if necessary, for example, until the subject experiences stable disease or regression, or until the subject experiences disease progression or unacceptable toxicity. Stable disease or lack thereof is determined by methods known in the art such as evaluation of subject's symptoms, physical examination, or diagnostic testing.

In certain embodiments, the combination regimen is administered to the subject over an extended period of time, ranging from about 1 day to about 50 years, from about 10 days to about 25 years, from about 1 month to about 10 years, or from about 6 months to about 5 years. In certain embodiments, the combination regimen is administered to the subject for about 12 weeks. In certain embodiments, the combination regimen is administered to the subject for about 6 months. In certain embodiments, the combination regimen is administered to the subject for about 1 year. In certain embodiments, the combination regimen is administered to the subject for about 2 years.

In certain embodiments, the combination regimen is cyclically administered to the subject. Cycling therapy involves the administration of the combination regimen provided herein for a period of time, followed by a rest for a period of time, and repeating this sequential administration.

As used herein, the term “combination regimen” includes the use of more than one therapies. However, the use of the term “combination regimen” does not restrict the order in which therapies (e.g., prophylactic and/or therapeutic agents) are administered to the subject. A first therapy (e.g., a prophylactic or therapeutic agent such as vitamin C provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as vitamin K provided herein) to the subject.

The methods provided herein may further comprise administering an additional therapeutic agent useful in treating, preventing, or ameliorating one or more symptoms of a polycystic disease. Effective dosages of the additional therapeutic agent can be administered together with, alternatively to, or sequentially to the administration of vitamins C and K. The dosages given will depend on absorption, inactivation, and excretion rates of the therapeutic agents as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Examples of the additional therapeutic agent include, but are not limited to, anti-atherosclerotic agents, such as ACAT inhibitors; antibiotics, such as anthracyclines, bleomycins, mitomycin, dactinomycin, and plicamycin; anticoagulants, such as acenocoumarol, argatroban, bivalirudin, lepirudin, fondaparinux, heparin, phenindione, warfarin, and ximelagatran; antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, indomethacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; antiproliferatives, such as methotrexate, FK506 (tacrolimus), and mycophenolate mofetil; anti-TNF antibodies or soluble TNF receptor, such as etanercept, rapamycin, and leflunimide; aP2 inhibitors; beta-adrenergic agents, such as carvedilol and metoprolol; bile acid sequestrants, such as questran; calcium channel blockers, such as amlodipine besylate; chemotherapeutic agents; bisphosphonates, such as alendronate, risendronate, ibandtonate, pamidronate, and etidronate; cyclooxygenase-2 (COX-2) inhibitors, such as celecoxib and rofecoxib; cyclosporins; cytotoxic drugs, such as azathioprine and cyclophosphamide; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; endothelin converting enzyme (ECE) inhibitors, such as phosphoramidon; enzymes, such as L-asparaginase; Factor VIIa inhibitors and Factor Xa inhibitors; farnesyl-protein transferase inhibitors; fibrates; growth factor inhibitors, such as modulators of PDGF activity; growth hormone secretagogues; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; hormonal agents, such as glucocorticoids (e.g., hydrocortisone and cortisone), estrogens/antiestrogens, androgens/antiandrogens, progestins, luteinizing hormone-releasing hormone antagonists, and octreotide acetate; pasireotide; immunosuppressants; mineralocorticoid receptor antagonists, such as spironolactone and eplerenone; microtubule-disruptor agents, such as ecteinascidins; microtubule-stabilizing agents, such as pacitaxel, docetaxel, and epothilones A-F; MTP inhibitors; niacin; phosphodiesterase inhibitors, such as PDE III inhibitors (e.g., cilostazol) and PDE V inhibitors (e.g., sildenafil, tadalafil, and vardenafil); plant-derived products, such as vinca alkaloids, epipodophyllotoxins, and taxanes; platelet activating factor (PAF) antagonists; platinum coordination complexes, such as cisplatin, satraplatin, and carboplatin; potassium channel openers; prenyl-protein transferase inhibitors; protein tyrosine kinase inhibitors; protein serine/threonine inhibitors; renin inhibitors; squalene synthetase inhibitors; steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; TNF-alpha inhibitors, such as tenidap; thrombin inhibitors, such as hirudin; thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); thromboxane receptor antagonists, such as ifetroban; topoisomerase inhibitors; vasopeptidase inhibitors (dual NEP-ACE inhibitors), such as omapatrilat and gemopatrilat; and other miscellaneous agents, such as, hydroxyurea, procarbazine, mitotane, hexamethylmelamine, and gold and silver compounds.

In certain embodiments, provided herein is a method for inhibiting cystogenesis in an organ, comprising contacting the organ with an effective amount of vitamin C, or a pharmaceutically acceptable salt, solvate, or hydrate thereof; and vitamin K, or a single enantiomer, a mixture of enantiomers, or a mixture of diastereomers thereof, or a pharmaceutically acceptable salt, solvate, or hydrate thereof.

In certain embodiments, the organ is a kidney. In certain embodiments, the organ is a liver.

The combination regimes provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes containers and dosage forms of the compounds in the combination regimens provided herein.

In certain embodiments, the kit includes a container comprising dosage forms of the compounds in the combination regimens provided herein, in one or more containers.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

### Animals and Cell Culture

Animals (rats and mice) were maintained on a standard diet after Mayo Institutional Animal Care and Use Committee approval. They were anesthetized with pentobarbital (50 mg/kg). Blood was collected from PCK rats by cardiac puncture. Liver and kidneys were fixed and embedded in paraffin for histology. For in vitro study, cholangiocytes were isolated from normal and PCK rats and cultured according to the procedures as described in Banales et al., Hepatology 2009, 49, 160-74. Normal and diseased human liver tissue were obtained from Mayo Clinical Core and National Disease Research Interchange.

Flow Cytometry:

Normal (n=5) and PCK cholangiocytes (n=6) were fixed in ethanol and suspended in 50 μg/mL propidium iodide containing 0.1 mg/mL RNase. Cell cycle analysis was performed at Mayo Advanced Genomics Technology Center.

Immunofluorescence Confocal Microscopy:

Microscopy was performed with Zeiss LSM-510 microscope (Carl Zeiss, Thornwood, N.Y.) using liver tissue of control and PCK rats; control and Pkd2^{WS25/-} mice; healthy human beings and patients with ADPKD, ARPKD and CHF. Liver sections were stained with primary antibodies against PCNA and Cdc25A (Santa Cruz Biotechnology, Santa Cruz, Calif.; 1:100). Respective secondary antibodies (Invitrogen, Carlsbad, Calif.; 1:200) were applied. Apoptosis was assessed by Terminal deoxynucleotidyl transferase dUTP nick-end labeling (TUNEL) assay (Chemicon, Billerica, Mass.). Mitotic and apoptotic indices were calculated, correspondingly, as a percent of PCNA- or TUNEL-positive nuclei out of 500 cells in randomly selected fields of liver and kidney sections.

Western Blot:

For western blotting: (i) cholangiocytes isolated from control and PCK rats; control and Pkd2^{WS25/-} mice; healthy human beings and ADPKD patients; and (ii) cultured PCK cholangiocytes were used. The cholangiocytes were resuspended in RIPA Buffer (Santa Cruz). Protein (30 μg) was run in 4-15% Tris-HCl sodium dodecyl sulfate-polyacrylamide gel, transferred to a membrane (BioRad, Hercules, Calif.), and incubated overnight at 4° C. with antibodies against PCN (1:500). Respective secondary antibodies (Invitrogen; 1:5000) were applied for 60 min. Bands were visualized with the ECL Plus Western Blotting Detection Kit (BD Biosciences). Actin staining was normalized for protein loading (Abcam; 1:1000).

### Example 1

### Comparison of Cystic Cholangiocytes with Normal Cholangiocytes

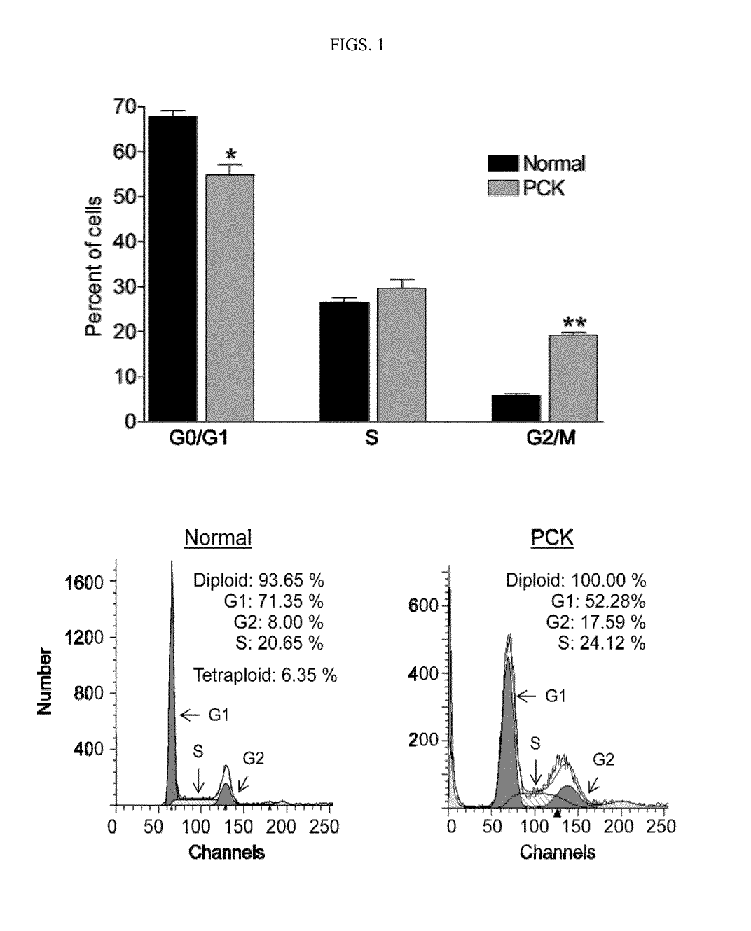

In cystic cholangiocytes isolated from an animal model of PLD/PKD, PCK rats, the percent of cells in G0/G1 phase was reduced, the percent of cells in S phase was not altered, and the percent of cells in G2/M phase was increased, in comparison with normal cholangiocytes (FIG. 1).

As shown in FIG. 1A, the majority of cells in normal rat cholangiocytes (n=5) were present in G0/G1 phase and the percent of cholangiocytes in G2/M phase was relatively low. In PCK cholangiocytes (n=6), the percent of cells in G0/G1 phase was decreased, while the percent of cells in G2/M phase was increased compared to normal. As shown in FIG. 1B, the cultured normal and PCK cholangiocytes were both diploid.

The rate of proliferation in humans and rodents was also examined by determining proliferating cell nuclear antigen (PCNA) expression. PCNA was overexpressed in cystic cholangiocytes. In normal human, rat, and mouse cholangiocytes (n=3 of each), PCNA expression (green) was nearly absent. Cystic cholangiocytes in human patients with ARPKD (n=3), ADPKD (n=5), CHF (n=3); in PCK rats (n=6) and Pkd2^{WS25/-} mice (n=5) showed a rigorous PCNA staining. In other words, cystic cholangiocytes in patients with PLD/PKD, in PCK rats, and Pkd2^{WS25/-} mice were PCNA immunoreactive, whereas normal cholangiocytes were not PCNA immunoreactive.

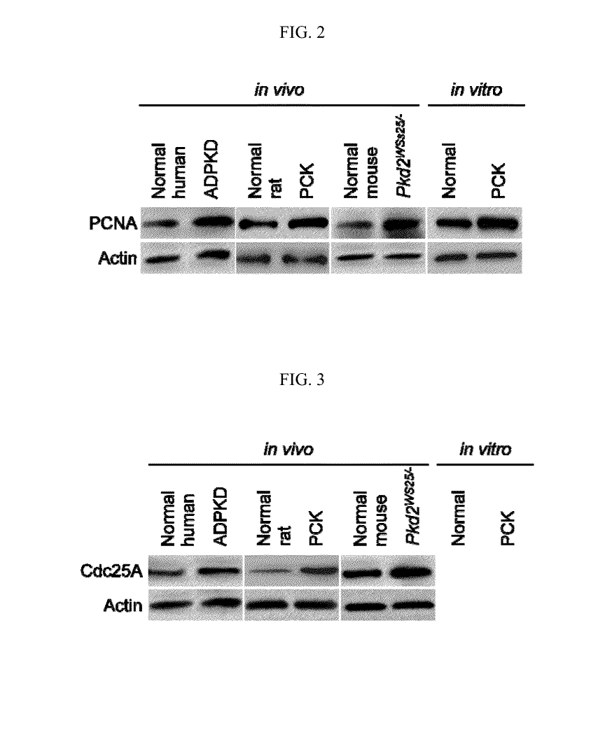

Western blots (n=3 for each) further confirmed that PCNA levels were increased in vivo in cholangiocytes isolated from ADPKD patients, PCK rats, and Pkd2^{WS25/-} mice, and in vitro in PCK-derived cholangiocytes compared to respective normal controls (FIG. 2).

Cell division cycle 25 homolog A (Cdc25A) was found to be over-expressed in cystic cholangiocytes. Relatively low expression of Cdc25A was found in normal human (n=5), rat (n=5), and mouse (n=4) cholangiocytes as measured using confocal microscopy (×40). Cdc25A was found to be increased in cystic cholangiocytes of patients with ARPKD, ADPKD, CHF, in PCK rats, and Pkd2^{WS25/-} mice (n=5 of each). As shown in FIG. 3, western blots (n=3 for each set of data) demonstrated that Cdc25A levels were elevated in vivo and in vitro compared to normal controls, respectively.

### Example 2

### Treatment of Polycystic Diseases with APATONE®

Thirty seven PCK rats (20 females and 17 males) at age of 3 weeks were divided into four groups: (i) vitamin C (VC) treatment group: 5 females and 4 males; (ii) vitamin K₃ (VK3) treatment group: 5 females and 4 males; (iii) APATONE® treatment group: 6 females and 5 males; and (iv) control group: 4 females and 4 males. Similarly, twenty Pkd2^{WS25/-} mice (12 females and 8 males) at age of 5 months were divided into four groups: (i) vitamin C (VC) treatment group: 3 females; (ii) vitamin K₃ (VK3) treatment group: 2 females and 2 males; (iii) APATONE® treatment group: 4 females and 3 males; and (iv) control group: 3 females and 3 males.

The VC treatment groups were given vitamin C at a concentration of 15 g/L in drinking water. The VK3 treatment groups were given vitamin K₃ at a concentration of 0.15 g/L in drinking water. The APATONE® treatment groups were given vitamins C and K3 at concentrations of 15 g/L and 0.15 g/L, respectively, in drinking water. The control groups were given drinking water only. Rats or mice in each group were allowed to drink freely.

The rats and mice were sacrificed after 6 weeks of treatment. The following parameters were analyzed: body weights, liver and kidney weights, serum biochemistry, renal and hepatic cystic and fibrotic areas, apoptotic and proliferation indices, expression of Cdc25A and its down-stream targets.

Since no differences was observed in liver and kidney weights between male and female PCK rats and Pkd2^{WS25/-} mice, male and female data were combined for statistical analysis. Cystic and fibrotic areas were analyzed according to the procedures as described in Masyuk et al., Gastroenterology 2007, 132, 1104-1116. Briefly, livers and kidney sections were stained with H&E or picrosirius red collagen to assess, respectively, cystic or fibrotic areas. Measurements were done by Meta-Morph software (Universal Imaging, West Chester, Pa.), following image acquisition using a light microscope and color digital camera (Nikon DXM 1200). Hepatic and renal cystic and fibrotic areas were expressed as a percent of total hepatic or renal parenchyma, respectively.

The effect of APATONE® on liver and kidney weights was evaluated in vivo using PCK rats, an animal model of one of PLD/PKD, ARPKD. As summarized in Tables 1 and 2, no visible defects were observed in treated anaimals. Treatment with APATONE® decreased liver and kidney weights in PCK rats, with APATONE® being more effective than VK3 alone.

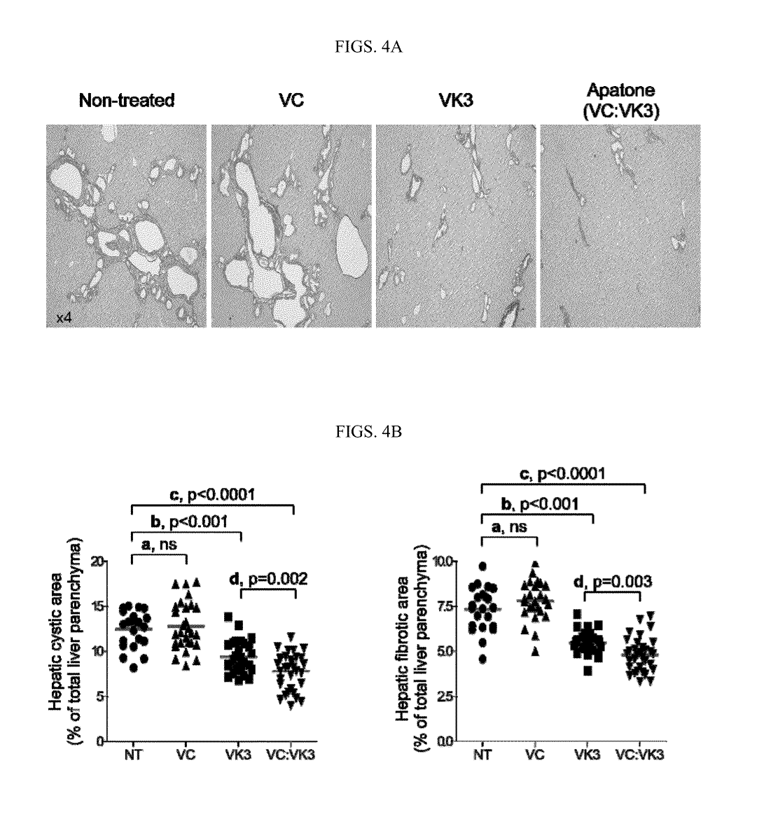

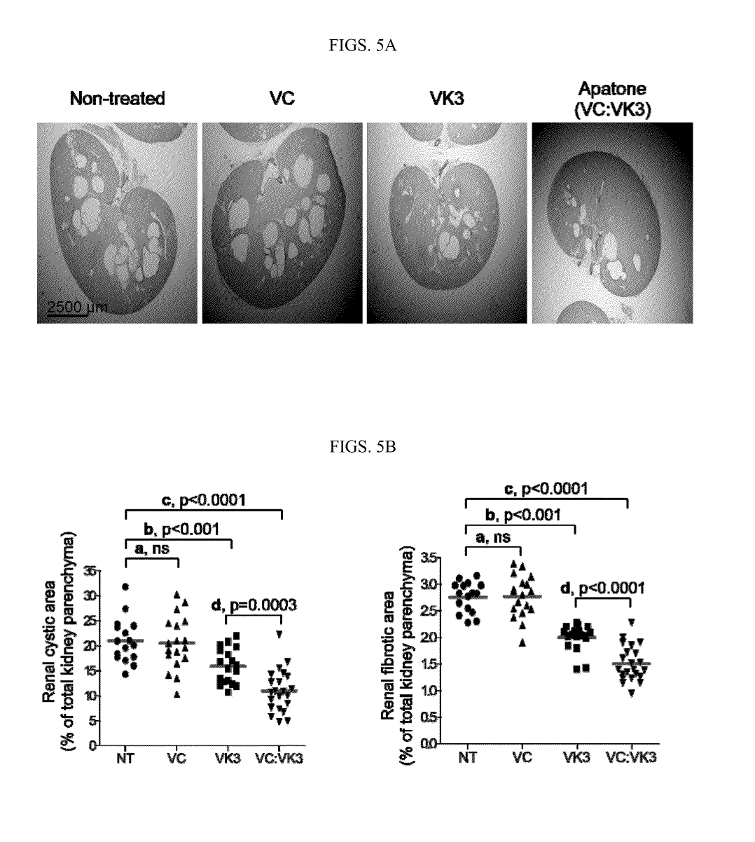

The effect of APATONE® on hepatic cystogenesis was evaluated in vivo using PCK rats. As shown in FIGS. 4 and 5, APATONE® descreased hepatic cystic and fibrotic areas of PCK rats comparied to non-treated counterparts. APATONE® suppressed hepatic and renal cystogenesis more effectively than VK3 alone.

The effect of APATONE® on liver and kidney weights was evaluated in vivo using Pkd2^{WS25/-} mice, which is an animal model of ARPKD. As summarized in Tables 3 and 4, treatment with APATONE® decreased liver and kidney weights in Pkd2^{WS25/-} mice with APATONE® being more effective than VK3 alone.

TABLE 1

| | Control | VC | VK3 | APATONE ® |
|---|---|---|---|---|
| Body Weight (g) | | | | |
| Male | 412 ± 15 | 416 ± 13 | 408 ± 16 | 423 ± 13 |
| Female | 270 ± 7 | 261 ± 10 | 276 ± 9 | 281 ± 15 |
| Liver Weight (g) | | | | |
| Male | 23.4 ± 0.9 | 23.3 ± 1.8 | 20.8 ± 0.5 | 19.6 ± 0.8 |
| Female | 17.0 ± 1.0 | 16.6 ± 1.3 | 15.5 ± 0.6 | 14.7 ± 0.6 |
| Liver | | | | |
| Weight (% bw) | | | | |
| Male | 5.68 ± 0.17 | 5.61 ± 0.14 | 5.12 ± 0.09 | 4.63 ± 0.14 |
| Female | 6.31 ± 0.15 | 6.36 ± 0.23 | 5.60 ± 0.13 | 5.12 ± 0.11 |
| Kidney Weight (g) | | | | |
| Male | 4.89 ± 0.12 | 4.95 ± 0.18 | 4.57 ± 0.13 | 4.28 ± 0.14 |
| Female | 3.99 ± 0.14 | 3.88 ± 0.19 | 3.78 ± 0.09 | 3.46 ± 0.12 |
| Kidney | | | | |
| Weight (% bw) | | | | |
| Male | 1.19 ± 0.09 | 1.19 ± 0.13 | 1.11 ± 0.11 | 1.04 ± 0.06 |
| Female | 1.48 ± 0.18 | 1.47 ± 0.11 | 1.36 ± 0.13 | 1.21 ± 0.09 |

TABLE 2

| | | VK3 vs | APATONE ® | APATONE ® |
|---|---|---|---|---|
| | VC vs Ctrl | Ctrl | vs Ctrl | vs VK3 |
| Body Weight | | | | |
| (g) Change | | | | |
| Male | 0.8% | −1.1% | 2.6% | 3.8% |
| Female | −3.3% | 2.2% | 3.6% | 1.8% |
| Liver Weight | | | | |
| (g) Change | | | | |
| Male | −0.4% | −10.9%* | −16.4%* | −6.2% |
| Female | −2.8% | −9.2%* | −14.0%* | −5.3% |
| Liver Weight | | | | |
| (% bw) Change | | | | |
| Male | −1.2% | −9.8%* | −18.5%* | −9.8%* |
| Female | 0.8% | −11.3%* | −18.7%* | −8.6%* |
| Kidney Weight | | | | |
| (g) Change | | | | |
| Male | 1.2% | −6.5%* | −12.5%* | −6.4%* |
| Female | −2.8% | −5.2%* | −13.3%* | −8.5%* |
| Kidney Weight | | | | |
| (% bw) Change | | | | |
| Male | 0 | −6.7%* | −12.6%* | −6.5%* |
| Female | −0.7% | −8.1%* | −18.2%* | −11.0%* |

*p ≦ 0.05

TABLE 3

| | Control | VC | VK3 | APATONE ® |
|---|---|---|---|---|
| Body Weight (g) | 26.3 ± 1.2 | 26.7 ± 2.0 | 27.9 ± 1.9 | 26.6 ± 0.9 |
| Liver Weight (g) | 2.12 ± 0.11 | 2.29 ± 0.43 | 1.64 ± 0.15 | 1.05 ± 0.13 |
| Liver Weight (%) | 8.05 ± 0.16 | 9.29 ± 2.01 | 5.87 ± 0.36 | 4.45 ± 0.21 |
| Kidney Weight (g) | 0.49 ± 0.02 | 0.46 ± 0.04 | 0.42 ± 0.01 | 0.32 ± 0.02 |
| Kidney Weight (%) | 1.89 ± 0.11 | 1.76 ± 0.16 | 1.53 ± 0.05 | 1.38 ± 0.06 |

TABLE 4

| | VC vs | VK3 vs | APATONE ® | APATONE ® |
|---|---|---|---|---|
| | Ctrl | Ctrl | vs Ctrl | vs VK3 |
| Body Weight (g) | 1.5% | 5.8% | −3.0% | −8.3% |
| Change | | | | |
| Liver Weight (g) | 8.0% | −22.6%* | −50.5%* | −36.0%* |
| Change | | | | |
| Liver Weight | 15.4% | −27.1%* | −44.7%* | −24.2%* |
| (% bw) Change | | | | |
| Kidney Weight (g) | −6.1% | −14.3%* | −34.7%* | −2.38%* |
| Change | | | | |
| Kidney Weight | −6.7% | −19.0%* | −27.0%* | −9.8%* |
| (% bw) Change | | | | |

*p ≦ 0.05

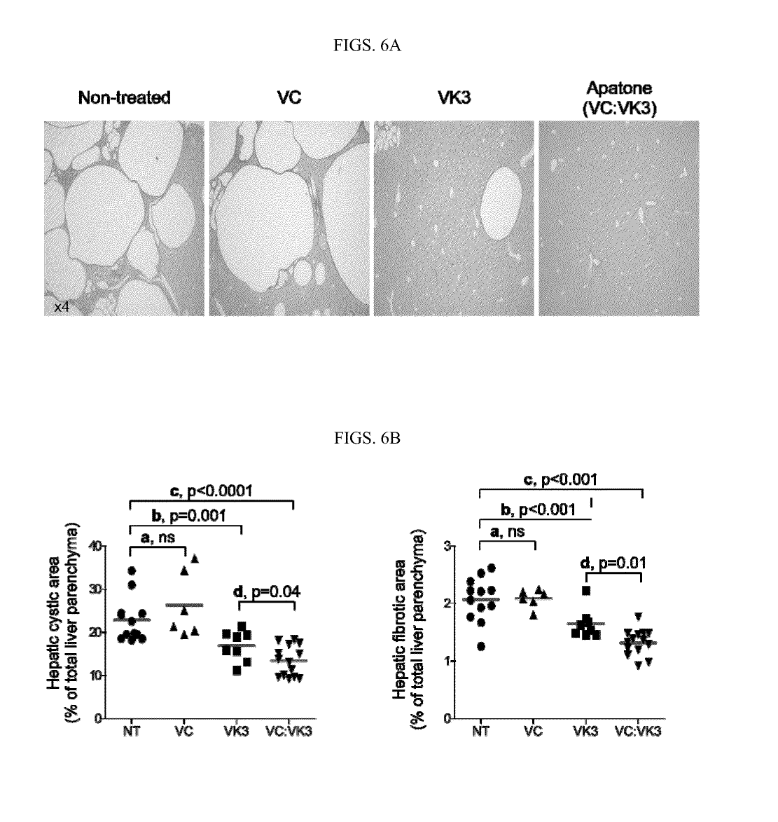

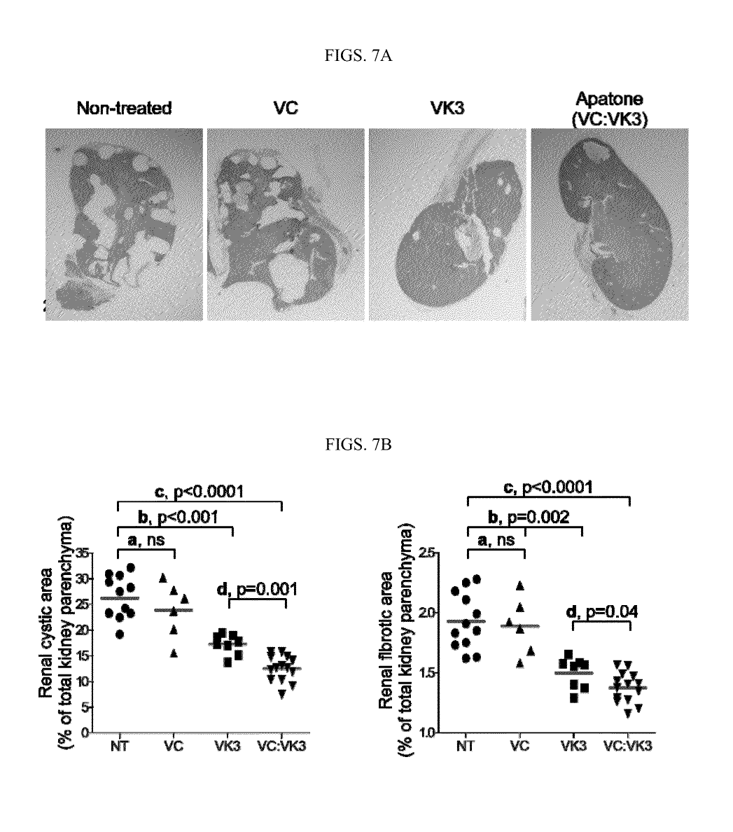

The effect of APATONE® on hepatic cystogenesis was evaluated in vivo using Pkd2^{WS25/-} mice. As shown in FIGS. 6 and 7, APATONE® descreased hepatic cystic and fibrotic areas of Pkd2^{WS25/-} mice comparied to non-treated counterparts. APATONE® suppressed hepatic and renal cystogenesis more effectively than VK3 alone.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, preventing, or ameliorating a polycystic disease in a subject, comprising administering to the subject (i) vitamin C, or a pharmaceutically acceptable salt, solvate, or hydrate thereof and (ii) vitamin K, or a single enantiomer, a mixture of enantiomers, or a mixture of diastereomers thereof, or a pharmaceutically acceptable salt, solvate, or hydrate thereof.

2. The method of claim 1, wherein the polycystic disease is a polycystic kidney disease.

3. The method of claim 1, wherein the polycystic disease is a polycystic liver disease and a polycystic kidney disease.

4. The method of claim 1, wherein the polycystic disease is an autosomal dominant polycystic kidney disease.

5. The method of claim 1, wherein the polycystic disease is a polycystic kidney disease caused by a mutation in PKD1.

6. The method of claim 1, wherein the polycystic disease is a polycystic kidney disease caused by a mutation in PKD2.

7. The method of claim 1, wherein the polycystic disease is a polycystic kidney disease caused by a mutation in PKD3.

8. The method of claim 1, wherein the polycystic disease is an autosomal recessive polycystic kidney disease.

9. The method of claim 1, wherein the polycystic disease is a polycystic kidney disease caused by a mutation in PKHD1.

10. The method of claim 1, wherein the polycystic disease is a polycystic liver disease.

11. The method of claim 1, wherein the polycystic disease is a polycystic liver disease caused by a mutation in PPRKCSH.

12. The method of claim 1, wherein the polycystic disease is a polycystic liver disease caused by a mutation in SEC63.

13. The method of claim 1, wherein the polycystic disease is a polycystic pancreas disease.

14. The method of claim 1, wherein the polycystic disease is a polycystic ovarian syndrome.

15. The method of claim 1, wherein the subject is a human.

16. The method of claim 1, wherein vitamin C is administered orally.

17. The method of claim 1, wherein vitamin C is administered topically.

18. The method of claim 1, wherein vitamin K is administered orally.

19. The method of claim 1, wherein vitamins C and K are administered together in a single composition comprising vitamin C, or a pharmaceutically acceptable salt, solvate, or hydrate thereof, and vitamin K, or a single enantiomer, a mixture of enantiomers, or a mixture of diastereomers thereof, or a pharmaceutically acceptable salt, solvate, or hydrate thereof.

20. The method of claim 19, wherein the composition is formulated in a single oral dosage form.

21. The method of claim 20, wherein the single oral dosage form is provided as a tablet or capsule.

22. The method of claim 20, wherein the single oral dosage form is provided as a capsule.

23. The method of claim 19, wherein the composition comprises about 500 mg of vitamin C, or a pharmaceutically acceptable salt, solvate, or hydrate thereof; and about 5 mg of vitamin K, or a single enantiomer, a mixture of enantiomers, or a mixture of diastereomers thereof, or a pharmaceutically acceptable salt, solvate, or hydrate thereof.

24. The method of claim 19, wherein the composition consists essentially of vitamin C, or a pharmaceutically acceptable salt, solvate, or hydrate thereof, in combination with vitamin K, or a single enantiomer, a mixture of enantiomers, or a mixture of diastereomers thereof, or a pharmaceutically acceptable salt, solvate, or hydrate thereof.

25. The method of claim 1, wherein vitamin K is vitamin K₃.

26. The method of claim 25, wherein vitamin K is 1,2,3,4-tetrahydro-2-methyl-1,4-dioxo-2-naphthalenesulfonic acid or a pharmaceutically acceptable salt thereof or a pharmaceutically acceptable solvate or hydrate thereof.

27. The method of claim 25, wherein vitamin K is an alkali or alkaline earth metal salt of 1,2,3,4-tetrahydro-2-methyl-1,4-dioxo-2-naphthalenesulfonic acid, or a pharmaceutically acceptable solvate or hydrate thereof.

28. The method of claim 25, wherein vitamin K is sodium or magnesium 1,2,3,4-tetrahydro-2-methyl-1,4-dioxo-2-naphthalenesulfonate, or a pharmaceutically acceptable solvate or hydrate thereof.

29. The method of claim 25, wherein vitamin K is anhydrous sodium 1,2,3,4-tetrahydro-2-methyl-1,4-dioxo-2-naphthalenesulfonate.

30. The method of claim 1, wherein vitamin C is L-ascorbic acid or a pharmaceutically acceptable salt thereof, or a pharmaceutically acceptable solvate or hydrate thereof.
